(19) United States
(12) Patent Application Publication
(10) Pub. No.: US 2004/0219659 A1
(43) Pub. Date: Nov. 4, 2004

(54) Multi-dimensional strain bioreactor

(76) Inventors: Gregory H. Altman, Dedham, MA (US); Gordana Vunjak-Novakovic, Belmont, MA (US); Peter R. H. Stark, Andover, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/421,445

(22) Filed: Apr. 22, 2003

Related U.S. Application Data
(60) Provisional application No. 60/375,096, filed on Apr. 22, 2002.

(51) Int. Cl. C12M 3/00

(52) U.S. Cl. 435/284.1; 435/299.1; 435/289.1; 435/293.1; 623/915; 623/916

(57) ABSTRACT

The invention features a bioreactor system. The system includes components, which exert physiologically relevant translational and rotational strains on a growing bioengineered tissue.

This application claims priority to provisional application U.S. Ser. No. 60/375,096, filed on Apr. 22, 2002, the entire contents of which is hereby incorporated by reference.

### TECHNICAL FIELD

This invention relates to tissue bioengineering.

### BACKGROUND OF THE INVENTION

Every year more than 135,000 Americans tear or rupture their anterior cruciate ligament (ACL) (Chen et al., J. Biomed. Mat. Res. 14: 567-586 (1980); Butler, D. L., J. Orthop. Res. 7: 910-921 (1989); Langer et al., Science 260: 920-926 (1993)). The ACL serves as a primary stabilizer of anterior tibial translation and as a secondary stabilizer of valgus-varus knee angulation, and is often susceptible to rupture or tear resulting from a flexion-rotation-valgus force associated with sports injuries and traffic accidents. Ruptures or tears often result in severe limitations in mobility, pain and discomfort, and the loss of an ability to participate in sports and exercise. Failures of the ACL are classified in three categories: (1) ligamentous (ligament fibers pull apart due to tensile stress), (2) failure at the bone-ligament interface without bone fracture, and (3) failure at the bone-ligament interface with bone fracture at the attachment site of bone and ligament. The most common type of ACL failure is the first category, ligamentous.

Bioreactors are a key component of tissue engineering for replacement tissues such as ligaments, and mechanical stress plays a significant role in tissue formation and repair. However, accurate simulation physiological conditions in vitro has been difficult to achieve.

### SUMMARY OF THE INVENTION

The invention features a bioreactor system and method for producing bioengineered tissue, e.g., an ACL, as well as a variety of other tissue types that require mechanical deformation, fluidic flux, or biochemical signalling for function. Such tissues include tendons, bones, cartilage, and blood vessels. The system includes components, which exert physiologically relevant translational and rotational strains on a growing tissue engineered prothesis. Application of such strains is required for growth and differentiation of cells into functional tissue structures and proper development of a structure suitable for function and capable of withstanding physiological forces following implantation in vivo.

The bioreactor includes advanced control elements for the cultivation of tissue in a mechanically simulated environment. The method utilizes the bioreactor for the cultivation of pluripotent stem cells in a three dimensional matrix under conditions appropriate for cell growth and regeneration, while subjecting the matrix to one or more mechanical forces via movement of one or a pair anchors to which the matrix is attached.

In general, the invention provides a bioreactor system for producing bioengineered tissue such as a ligament or tendon tissue using a tissue matrix. The tissue matrix is tubular in shape. Alternatively, the matrix is configured in any shape through which tissue culture media can be perfused. The matrix is preferably a porous or fibrous composition such as silk. The system includes a reactor vessel configured to define an interior vessel volume to contain the tissue matrix and a tissue culture medium for providing nutrients to the tissue matrix, the vessel having a first portion configured to couple to the tissue matrix at a location opposite a hole defined by a second portion of the vessel, the first portion providing a first vessel passageway in fluid communication with the interior volume of the vessel and a first vessel port, a shaft configured to move within the hole defined by the second portion of the reactor vessel and to couple to the tissue matrix, the shaft providing a shaft passageway disposed to be in fluid communication with a shaft port and with an interior volume of the tissue matrix when the matrix is coupled to the shaft, and a pump coupled in series between the shaft port and the first vessel port and configured to produce a flow of the culture through the first portion of the vessel, the shaft, and the interior volume of the tissue matrix.

Implementations of the invention may include one or more of the following features. The vessel is configured such that the interior vessel volume has a substantially constant volume. The vessel has a substantially rigid exterior. The first portion of the vessel provides at least one second passageway in fluid communication with the interior vessel volume, the second portion of the vessel provides at least one third passageway in fluid communication with the interior vessel volume. The pump is coupled to the vessel to be in fluid communication with the at least one second passageway and the at least on third passageway to produce a flow of the culture in the interior vessel volume exterior to the tissue matrix. The system further includes a tension load monitor including first and second couplings, the first coupling being coupled to the reactor vessel in a fixed relationship to the location of the first portion of the reactor vessel that is configured to couple to the tissue matrix. The system further includes a culture chamber coupled in series with the first vessel port, the shaft port, and the pump, and configured to produce the culture such that the culture has a desired ratio of N₂, O₂, and CO₂ in the chamber.

One or more of the following features are optionally included in the bioreactor system. The shaft is coupled to the reactor vessel such that the shaft can slide within the hole in the second portion of the vessel and rotate with respect to the vessel substantially free of frictional forces between the shaft and the vessel while inhibiting exogenous items from entering the interior vessel volume. The system further includes a bellows coupled to the shaft and to the reactor vessel. The system further includes moving means for at least one of translating and rotating the shaft relative to the reactor vessel. The moving means is configured to translate and rotate the shaft relative to the reactor vessel concurrently.

Also within the invention is a bioreactor system for producing tissue such as a ligament or tendon tissue using a tissue matrix, which includes the following components: a reactor vessel configured to define an interior vessel volume to contain the tissue matrix and a tissue culture for providing nutrients to the tissue matrix, the vessel having a first portion configured to couple to the tissue matrix at a location opposite a hole defined by a second portion of the vessel; a shaft configured to move within the hole defined by the second portion of the reactor vessel and to couple to the tissue matrix; and a tension load monitor including first and second couplings, the first coupling being coupled to the reactor vessel in a fixed relationship to the location of the first portion of the reactor vessel that is configured to couple to the tissue matrix.

Implementations of the invention include one or more of the following features. The shaft is coupled to the reactor vessel such that the shaft can slide within the hole in the second portion of the vessel and rotate with respect to the vessel substantially free of frictional forces between the shaft and the vessel while inhibiting exogenous items from entering the interior vessel volume. The system further includes a bellows coupled to the shaft and to the reactor vessel. The system further includes moving means for at least one of translating and rotating the shaft relative to the reactor vessel. The moving means is configured to translate and rotate the shaft relative to the reactor vessel concurrently. The system further includes a housing, wherein the second coupling of the load monitor is coupled to the housing.

In yet another aspect, the invention provides a bioreactor system for producing a tissue using a tissue matrix, the system including the following elements: a reactor vessel having a substantially rigid exterior and configured to define a substantially constant-volume interior volume to contain the tissue matrix and a tissue culture medium for providing nutrients to the tissue matrix, the vessel having a first portion configured to couple to a first end of the tissue matrix at a location opposite a hole defined by a second portion of the reactor, the first and second portions providing at least one first and second reactor passageway, respectively, in fluid communication with the interior volume of the vessel, the at least one first reactor passageway being in fluid communication with at least one reactor intake port and the at least one second reactor passageway being in fluid communication with at least one outlet port; a shaft configured to slide within the hole defined by the second portion of the reactor vessel and to couple to a second end of the tissue matrix, the shaft providing a shaft passageway disposed to be in fluid communication with a shaft intake port, and in fluid communication with an interior volume of the tissue matrix when the matrix is coupled to the shaft; a load monitor coupled to the reactor vessel in a fixed relationship to the first portion of the reactor vessel, a culture chamber having an input coupled to the at least one outlet port and configured to produce the culture having a desired ratio of N₂, O₂, and CO₂ in the chamber, the chamber being further coupled to the at least one reactor intake port and the shaft intake port; and a pump coupled between the at least one outlet port and the at least one reactor intake port and the shaft intake port and configured to produce a flow of the culture from the chamber, through the shaft and the second portion of the reactor vessel, into the interior volume of the vessel and the interior volume of the tissue matrix, through the first portion of the reactor vessel, and back to the chamber. The shaft is coupled to the reactor vessel such that the shaft slides within the hole in the second portion of the vessel and rotate with respect to the vessel substantially free of frictional forces between the shaft and the vessel while inhibiting exogenous items from entering the interior volume of the reactor vessel.

Other features, objects, and advantages of the invention will be apparent from the description and drawings.

### BRIEF DESCRIPTION OF THE FIGURES

FIG. 1A is a diagram of a bioreactor system. “1” represents a means for exerting tension and/or torsion forces. “2” represents a means for circulating fluid (e.g., tissue culture medium) such as perfusion and/or sheath flow. “3” represents a means for biochemical control, e.g., the gas composition (O₂, CO₂, and/or N₂) of the medium, and “4” represents a means for controlling the temperature of the medium.

FIG. 1B is a photograph of a functioning bioreactor system. (a) denotes a peristaltic pump, (b) denotes an environmental gas chamber, and (c) denotes two bioreactors containing 24 vessels.

FIG. 1C is a diagram of a bioreactor system showing a means (“5”) to measure force on the tissue. The force monitoring system measures both applied and resistance loading directly at the tissue-anchor interface, e.g., by means of a bellows mounted externally relative to the reaction vessel.

FIG. 2A is a diagram of an exploded-view of a reactor vessel assembly.

FIG. 2B is a photograph of a reactor vessel containing an anchored silk fiber matrix during culture with seeded cells. Vessel ‘seats’ (a) within the fixed-position plates and vessel locking bar (b) are shown.

FIG. 3A is a photograph of a bioreactor system. Bioreactors (a) are shown with 24 loaded reactor vessels (b). A traveler (c) housing both rotational (in view) and translational gear trains, the high torque stepper motors (d), and the linear bearing (e) are also shown.

FIG. 3B is a graph illustrating empirical translational and rotational displacement data for a programmed 10 cycle 2 mm and 90° regime at 0.0167 Hz.

FIG. 4 is a photograph of an environmental chamber prior to closure to show the internal silicone hose coils and gas inlet distribution manifold.

FIG. 5 is a line graph showing data obtained from the bioreactor system. pH and PO₂ levels were measured and recorded during a 21 day experiment.

FIGS. 6A-B are scanning electron micrographs of cells seeded on the silk fiber scaffold. FIG. 6A shows cells on a scaffold 1 hr post-seeding prior to loading into the reactor, and FIG. 6B shows cell on a scaffold after culturing in the bioreactor for 14 days. The arrow indicates cell sheet and possible extra-cellular matrix formation.

FIGS. 7A-D are photomicrographs stained cells. FIG. 7A shows an H&E stain of mechanically stimulated collagen gel longitudinal section after 14 days of culture. FIG. 7B shows an H&E stain of longitudinal section of static control. FIG. 7C shows an H&E stain of mechanically stimulated gel cross-section. FIG. D shows an H&E stain of static (control) cross-section.

### DETAILED DESCRIPTION OF THE INVENTION

The present invention is based on the finding that the histomorphological properties of an in vitro produced bioengineered tissue generated from pluripotent cells within a matrix are affected by the direct application of mechanical force to the matrix during tissue generation.

A bioreactor houses developing tissue and exposes the tissue to stimuli (e.g. physical, chemical, and electromagnetic). Cell differentiation is influenced by such stimuli. Stimuli are often produced by surrounding cells, such as secreted factors, cell-cell contact, chemical gradients, and specific pH levels. Other more unique stimuli are experienced by more specialized types of tissues (e.g. the electrical stimulation of cardiac muscle). The application of such tissue specific stimuli in concert with the appropriate mechanical forces promotes differentiation of the cells into a tissue which more closely approximates the specific natural tissue.

The bioreactor and methods described herein are useful for producing an ACL ex vivo Cells capable of differentiating into ligament cells are grown under conditions which simulate the movements and forces experienced by an ACL in vivo through the course of embryonic development into mature ligament function. This is accomplished by the following steps: under sterile conditions, pluripotent cells are seeded within a three dimensional matrix, of cylindrical shape, which is comprised of a material to which the cells can adhere (e.g. collagen gel). The faces of the matrix cylinder are each attached to respective anchors, through which a range of forces are to be applied to the matrix. To facilitate force delivery to the matrix, it is preferable that the entire surface of each respective face of the matrix contact the face of the respective anchors. Anchors with a shape which reflects the site of attachment (e.g. cylindrical) are best suited for use in this method. Once assembled, the cells in the anchored matrix are cultured under conditions appropriate for cell growth and regeneration. The matrix is subjected to one or more mechanical forces applied through the attached anchors (e.g. via movement of one or both of the attached anchors) during the course of culture. Methods of seeding matrices and producing bioengineered tissues are described in U.S. Pat. No. 6,287,340, the contents of which is hereby incorporated by reference.

The bioreactor device generates fully functional bioengineered tissue such as ACLs. Tissue engineering using the device allows development of biologically based functional tissues in vitro for transplantation. A key feature in tissue engineering is the use of a bioreactor to provide an environment to control and direct cellular responses toward functional tissue formation in vitro. Components of control include biochemical and nutrient requirements to support cell proliferation and/or differentiation, sufficient nutrient and metabolite transport to and from the supernatant to the developing tissue, and support or anchoring for the cells and developing tissue. Environmental factors, such as mechanical stress, which are precisely controlled, play a significant role in tissue development.

A variety of bioreactors have been reported for use in a range of cell and tissue studies involving mechanical stress. A 2-D flexible membrane culture systems was used to examine the effects of cyclic strain directly on cells in monolayer (ref: tenascin paper) or in combination with a substrate. A 3-D rotating bioreactor was used to increase uniform cell seeding of 3-D scaffolds and used to mimic a microgravity environment. 3-D uniaxial cyclic loading via a piston has been developed, and 3-D dynamic shear and compression bioreactors have been used to stimulate tissue explants such as cartilage or bone. 3-D fluidic reactors with pulsatile flow have been used to induce smooth muscle cell alignment for blood vessel engineering. The presence of physical forces (e.g. dynamically fluctuating hydrodynamic shear and pressure) during bioreactor cultivation of engineered cartilage and blood vessels has led to improved structural and functional properties of engineered tissues. None of these bioreactors directly mimic in vivo the physiological environment necessary for growing tissues as faithfully as the improved bioreactor system described herein.

The bioreactor described herein imparts in a controlled manner complex mechanical forces to growing tissues in 3-D environments that directly mimic the physiological environment in vivo. ACL was used as a model tissue due to the clinical is need to address limitations of current reconstructive techniques associated with autologous ACL reconstruction with patellar or hamstring tendon. These limitations include donor site morbidity, lengthy rehabilitation periods, increased risk of tendonitis, and early onset of arthritis. To address these issues, an improved reactor design was constructed to impart complex mechanical signals to the growing cells. The physiologically relevant mechanical forces applied to cultures in vitro generate functionally useful tissues for in vivo clinical applications. The improved reactor design provides an environment in which mechanical stimuli that are physiological in nature, are presented in a 3-D tissue-like environment to induce cellular differentiation and de novo ligament formation in vitro.

The device was used to culture progenitor bone marrow stromal cells (BMSCs) in combination with a protein, e.g., silk, under conditions of controlled complex mechanical signaling. There are no reported growth or regulatory factors that have been shown to promote adult stem cell differentiation into ligament-like cells in vitro. Therefore, more traditional approaches to tissue engineering, based on the addition of exogenous growth factors, cannot be employed. Furthermore, while there are a number of studies based on the use of fibroblasts in bioreactors to generate ligament-like tissues, inducing adult stem cells to differentiate into various cell types, is a relatively new option to forming functional relevant tissues in vitro. However, this goal can only be achieved if suitable environmental signals can be provided to the cells to direct their differentiation path toward ACL-like cells and tissues. The bioreactor reliably mimics physiological physical forces, to achieve proper differentiation of cells to yield a functional ACL. Mechanical signaling using the bioreactor system, without exogenous growth factors, successfully resulted in the differentiation of BMSCs into ligament forming cells within a 3-dimensional collagen gel matrix

Bioreactor Design.

The bioreactor tube design provided an environment for the growth of a 4 cm long ligament when considering the anchors, and approximately 2 cm long extending between the anchors. The terminology used in this document will be defined as follows: (a) translation load along the longitudinal axis of the ligament—tension; (b) rotational load about the longitudinal axis of the ligament—torsion; (c) change in length (ΔLₜ) along the longitudinal axis of the ligament—linear deformation; (d) change in rotational degree (ΔL^{r}) about the longitudinal axis of the ligament—rotational deformation; (e) strain (ΔLₜ/Lₒₜ, where L₀ₜ=20 mm initial length of ligament) along the longitudinal axis of the ligament—translational strain; (f) strain (ΔLᵣ/Lₒᵣ, where L₀ᵣ=360° initial non-strained position of ligament) about the longitudinal axis of the ligament—rotational strain; (g) strain rate (ΔLₜ/Lₒₜ/time) along the longitudinal axis of the ligament—translational strain rate; (h) strain rate (ΔLᵣ/Lₒᵣ/time) about the longitudinal axis of the ligament—rotational strain rate; Note: strain is reported as a percentage of ΔL/Lₒ.

The reactor tubes and the apparatus were placed in an incubator at 37° C. with 5% CO₂. The reactor tubes are 2.54 cm in diameter and 10 cm long. The tubes were cut from Teflon stock tubing (McMaster-Carr Supply Co.). Each reactor tube was fitted with two nylon bulkhead-mounted luers which serve as ports for medium and gas exchange. The luers were fit within tapped holes to avoid protrusion into the inner area of the tube. The anchor mounts were machined from Teflon rod stock and a 12 mm diameter by 10 mm length hole was machined in the center of each anchor mount to allow for co-axial alignment of the coral anchors. The coral anchors were held in place with set-screws. The bottom section of the lower anchor mount and the lower translational plate, respectively, were machined with a square shape to prevent rotation of the reactor tube with respect to the translational plate. The cylindrical section of the lower anchor mount is inserted into the bottom of the teflon reactor tube and attached with a hose clamp. A stepper motor (Servo Systems, 400 steps/360°) coupled to a high precision lead screw (lead=0.635 mm/360°) and low drag torque anti-backlash nut mounted into the translational plate provide translational tolerances precise to 1.6 μm.

The upper anchor mount was attached to a rotational shaft with set screws. The shaft extended into the reactor tube through two teflon bearings. The lower of the two bearing was inserted into the top of the Teflon reactor tube and attached via a worm-drive clamp. The lower bearing did not move while allowing for the free rotation of the shaft. Super stretch silicone rubber thick was used to extend between the upper and lower teflon bearings in order to enclose the top of the reactor tube and provide a barrier against contamination.

The system used allowed for the application of a variety of loading regimens based on a combination of linear deformation (up to 2 mm and a 10% translational strain) and rotational strain (up to 25% and 90 degrees), with a collagen matrix which remained adherent to the coral anchors.

Cylindrical pieces of Goinopra coral, 12 mm in diameter and 20 mm in length with a pore size of 500 μm (supplied by Interpore-Cross International) were used as the anchors. The coral was treated by a hydrothermal process to convert the calcium carbonate to calcium phosphate (hydroxyapatite). This mineral content and pore size is similar to some types of human cancellous bone and this material has been approved by the FDA for bone grafts.

The coral anchors were fastened into the anchor mounts using the set screws. The upper and lower mounts, linear bearings, rotational shaft, and silicone membrane are assembled with the teflon tube. Two caps were placed on the luer ports and the reactor tube is autoclaved for 20 minutes. All materials were selected to be stable in the autoclave. After autoclaving, the upper luer cap is replaced with a Gelman Acrodisc CR PTFE 1.0 μm filter for gas exchange. The matrix and tissue culture medium containing the cells were injected through the lower port of the reactor tube using a 20 ml syringe. Following injection, the lower cap was replaced and the reactor tube inserted into the translational plate at a lowered position in the mechanical device. The translational plate was then raised so that the end of the rotational shaft extending from the reactor tube inserted into a linear bearing press fit into the rotational plate and a pin hub spur gear (120 teeth, 1.666 inch pitch diameter, Nordex) sitting above the plate. Once inserted into the gear, the rotational shaft was fastened with a set-screw. A second stepper motor coupled to a smaller pin hub spur gear (30 teeth, 0.4166 inch pitch diameter) was used to rotate the rotational shaft and hence the top coral anchor. Since the two gears (motor gear/rotation gear) are in a 4:1 ratio, tolerances precise to 0.225 degrees can be achieved with this device.

Control tubes consisted of identical components and conditions (cells, media, matrix, anchors) to those described for the bioreactor tube experimental set up with the exception that these tubes were not mechanically deformed (static) in the apparatus.

Software used to control the mechanical device was written using C programming language and Borland C++ Compiler Version 5.0. The mechanical device was designed specifically for periodic torsional and tensile loads along the longitudinal axis of the growing ligament. The software provided precise independent control over the rotational and linear movement and the rates of these movements. Rates for linear and rotational movement range from 1 mm/day and 90 degrees/day, respectively, to a maximum of 15 mm/sec and 90 degrees/sec. The software allowed the user to input the forward and return rotational and linear rates, the duration to reach and return from the extreme points (e.g., maximum angle and distance), an intermediate period of rest or static mode at the extreme point, a rest or static mode at the home point, and the number of repetitions for the cycle. Several different cycles with varying loading regimens are optionally programmed and run for the duration of the experiment. The software also allows the user to incorporate preset waveforms such as sinusoidal, square, triangular, and trapezoidal wave forms as well as ramp and hold at desired rates and amplitudes.

Mechanical Control and Production of a Functional Bioengineered Tissue

Bioreactors with advanced control of environmental conditions are essential for meeting the complex requirements of in vitro engineering of functional skeletal tissues.

A computer-controlled bench-top bioreactor system with capability to apply complex concurrent mechanical strains to three-dimensional matrices in conjunction with enhanced environmental and fluidic control was developed. The apparatus is applicable to any bodily tissue, e.g., skeletal tissue such as ligaments associated with skeletal joints, which is subject mechanical strain in vivo. For example, the system was used to make a tissue engineered ACL from human bone-marrow derived precursor cells, the differentiation of which is dependent upon exposure to complex mechanical and biochemical events. Environmental control of such mechanical and biochemical events is essential to proper differentiation of the cells and proper function of the engineered tissue. Precisely-controlled mechanical strains (at <0.1 mm for translational and <0.1° for rotational strain) were applied to developing tissue, while temperature was maintained at 37+/−0.2° C. (mean+/−standard deviation) about the developing tissue over the 21 days of operation. Twenty-four reactor vessels containing silk matrices seeded with hBMSCs maintained at constant 7.4 pH and 20% pO₂ over 21 days in culture and supported cell spreading and cell growth on silk fiber matrices as demonstrated by SEM at a flow rate of 2.5 ml/min, pH of 7.4+/−0.02 and pO₂ of 20+/−0.5%.

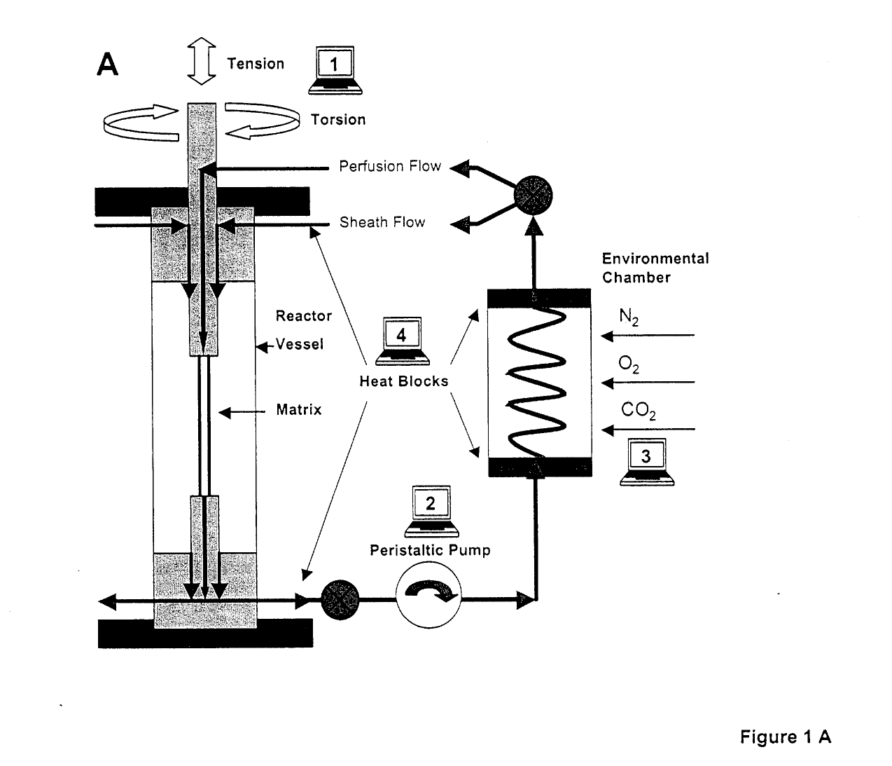

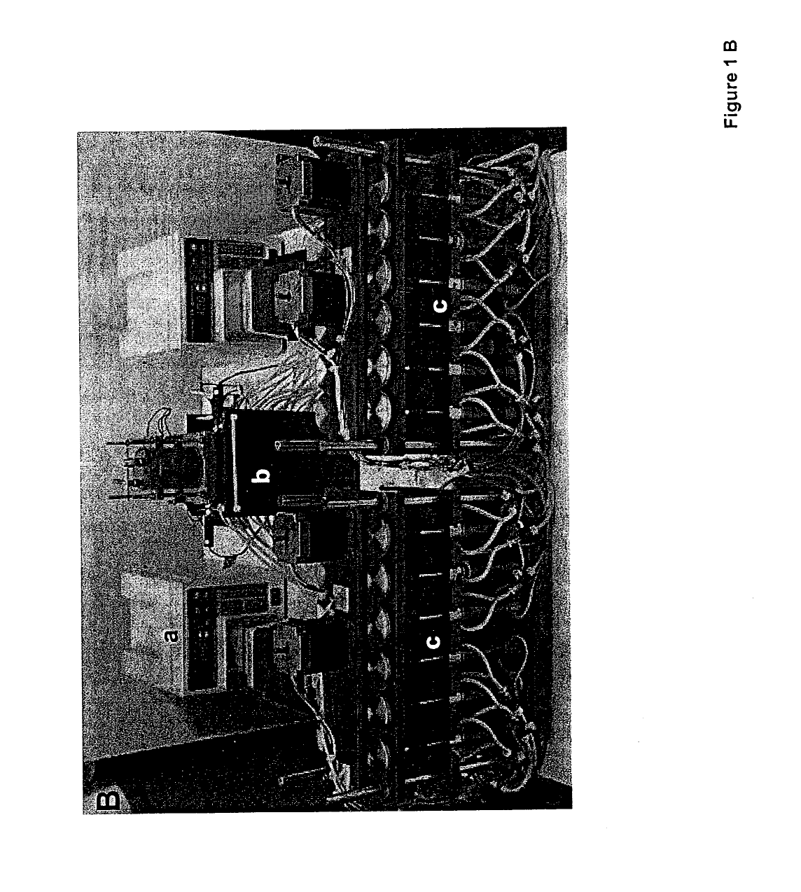

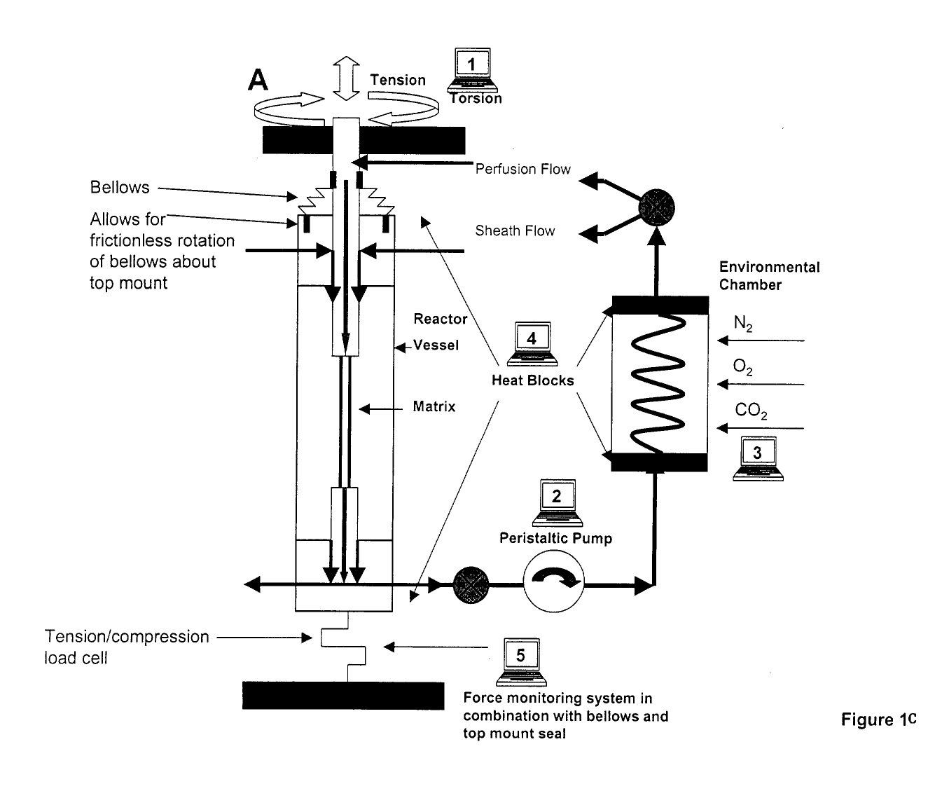

The system encompasses two independently controlled bioreactors that share a common environmental control chamber. The system, shown in FIGS. 1A-B, includes the following components/subsystems: (a) the reactor vessels in which the matrix is resident, (b) an environmental chamber to control gas exchange via the growth medium, (c) a multi-channel peristaltic pump to recirculate the growth medium, (d) a motion control subsystem to subject the matrix to mechanical stimuli and (e) a thermal control subsystem to maintain appropriate tissue growth temperatures. Each reactor vessel contains its own independent loop to avoid cross contamination and to allow change in biochemical factors from one vessel to another.

Bioreactor Vessel

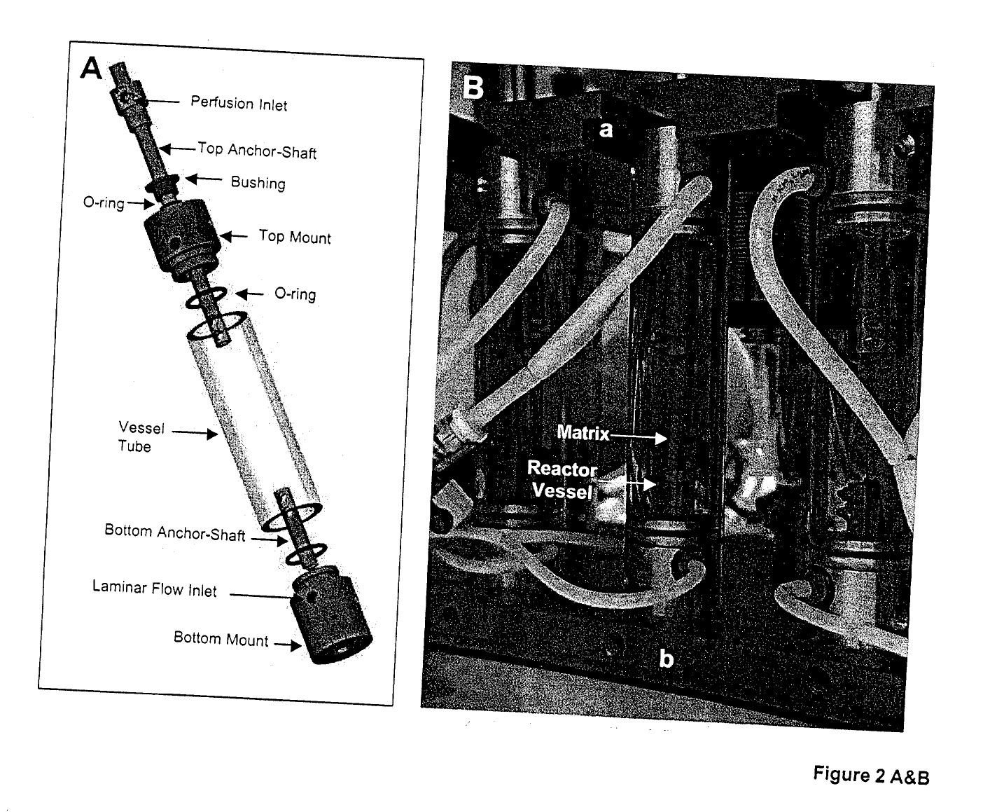

The bioreactor provides independent but concurrent control over translational and rotational strains imparted to the growing tissue housed within the reactor vessel. The co-axial reactor vessels provide at least two degrees of freedom for mechanical deformation of the growing ligaments. Each vessel contains a top and bottom mount, a vessel tube, and two anchor-shafts (FIG. 2A). Custom-designed parts are machined from 304 stainless-steel (S.S.). Off of the shelf items include: 18-8 S.S. set screws, polycarbonate tubing, nylon or kynar luer lock connections, bronze hubs and rubber Buna-O O-rings. The polycarbonate tubing, which comprises the vessel wall, is optically clear allowing real time observation. The inside diameter of the tube is fixed at 1.91 cm (0.75″) while the length of the tube is sized to accommodate the length of the matrix or tissue desired. Vessels allow for quick assembly/disassembly and all parts can be steam or gas sterilized.

Vessel Mounts

The top and bottom mounts provide two diametrically opposed radial inlets (or outlets). These feed 1.27 cm (0.5″) long annuli in the mounts. The annuli (of outer diameter of 0.95 cm (0.38″)) allow a radially invariant sheath flow around the 0.64 cm (0.25″) diameter shaft and into the main volume of the vessel. The annuli dimensions were calculated on the basis of the flow rates provided by the peristaltic pump (0.25-25 ml/min). When used to culture ligaments, laminar flow is achieved around the surface of the growing tissue by varing the flow rate. Both mounts employ O-ring grooves for sealing of the polycarbonate tube to the mounts (FIG. 2A). All inlet and outlet connections were made with nylon or kynar plastic luer lock fittings.

The bottom mount (FIG. 2A) contains a co-axial blind hole into which the anchor-shaft can be affixed via set-screws. The bottom mount also contains two blind threaded holes on the exterior, which do not protrude into the reactor vessel. One of the holes is radial while the other is coaxial. The radial hole is used to attach a stabilizing arm for use during vessel disassembly and while the axial hole for vessel loading into an Instron or a base-fixture. The top mount (FIG. 2A), designed with a 6.38 mm (0.25″) through hole and a counterbore at the top of the mount for an O-ring and bronze bushing, allows the top anchor-shaft to protrude from the interior of the vessel, through the O-ring and bushing, to the exterior of the vessel where it is coupled to the bioreactor for mechanical manipulation. The depth of the bushing into the counterbore controls the degree of compression of the O-ring against the top tissue anchor shaft, providing a barrier against contamination while allowing the shaft to move freely with two degrees of freedom, an axial motion and a rotational motion about that axis.

Vessel Sterile Barrier Seal

The top mount, designed with a 0.251″ hole and a “seat” at the top of the mount for an o-ring and bronze bushing, allows the top anchor-shaft to protrude from the interior of the vessel, through the o-ring and bushing, to the exterior of the vessel where it is attached to the bioreactor for mechanical manipulation. The depth of the bushing into the seat controls the degree of compression on the o-ring, providing a barrier against contamination while allowing the top anchor-shaft to move freely in two degrees of freedom, translation and rotation.

If force or load monitoring is desired, the o-ring, which introduced friction between the moving anchor shaft and top vessel mount is replaced with a silicone bellow external to the reactor vessel housing, i.e., the device lacks an o-ring. Through the appropriate choice of mount material and machining specifications (e.g., Teflon and a tolerance of +/−0.0005″) force resolution down to 1 Newton can be gained while still providing a barrier to contamination. Furthermore, the interface between the bellows and the anchor mount can be such that it allows for the free rotation of the bellows eliminating any torque in the system or bellows.

The resulting vessel designs provide a barrier to contamination while allowing for the free translation and rotation of the anchor shaft, which directly attaches to the tissue specimen housed within the reactor vessel. As a result, the system measures force through direct contact with the tissue with no change in reactor vessel volume. A change in reactor volume would result in a change in hydrostatic pressure or fluid flux that may adversely affect tissue growth or development. Change in pressure is undesirable, because it represents a variable in an otherwise controlled environment for the study desired tissue manipulations.

By decoupling the reactor vessel from the actuating bioreactor system, the overall system allows the tissue to be exposed to both rotational and translational strains, either independently or in combination while providing the ability to measure force directly at the tissue and provide a barrier to contamination.

Tissue Anchor-Shafts

The anchor-shafts (FIG. 2A) were machined from 304 S.S. tubing, (6.35 mm OD, 3.175 mm ID). The bottom and top anchor-shafts, 2.54 cm and 10.16 cm long, respectively, allow for fluid flow through their centers. The top anchor-shaft serves two functions, an external fluid inlet into the shaft system for perfusion flow through the tissue and coupling of the tissue/matrix to the mechanical manipulation. Two internally located 8-32 threaded holes and two clearance holes diametrically opposed distal from the tissue apart on the top and bottom anchors were provided to allow for a variety of tissue anchoring options (FIG. 2B). Fluidic control (0.25 ml/min to 25 ml/min) and pathways for perfusion through and/or sheath flow about the tissue were designed to increase possible cell seeding methodologies and support tissue growth and differentiation (FIGS. 1A & 2B).

Bioreactor Motion Control Subsystem

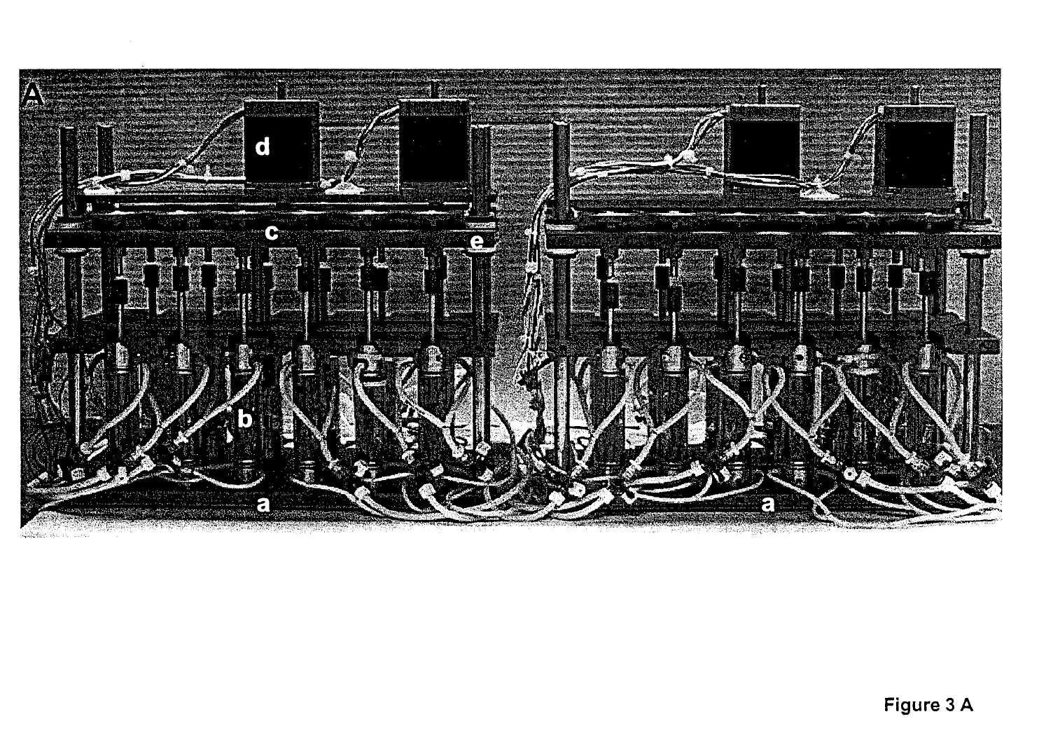

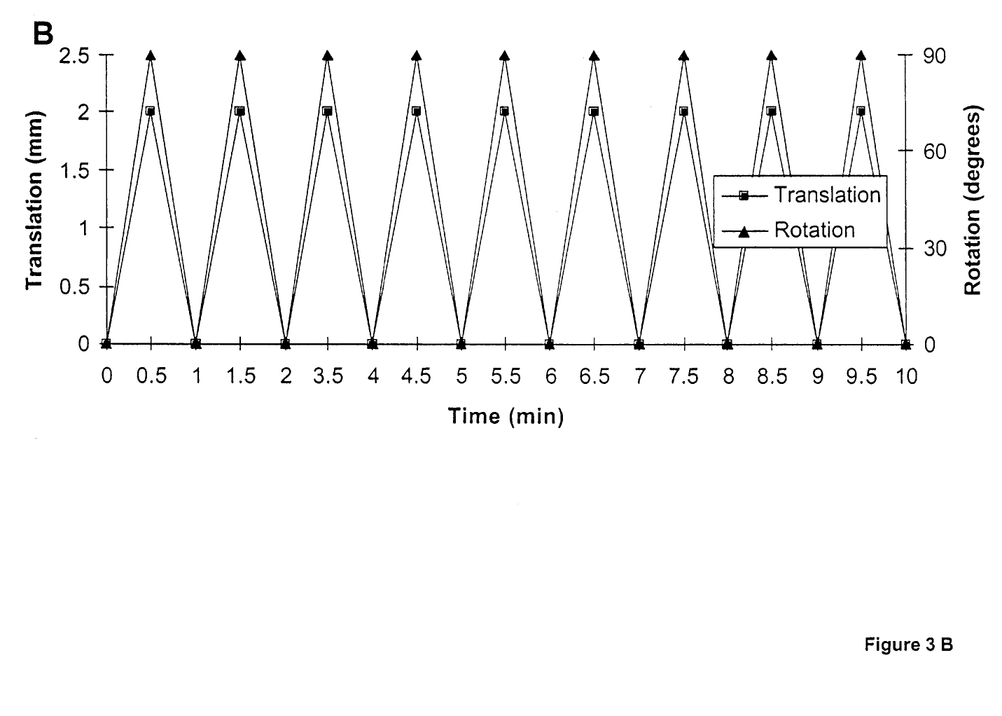

Each bioreactor houses two banks of six reactor vessels supporting the growth of up to 12 independent engineered ligaments (FIG. 3A). Each bioreactor contains two fixed-position aluminum plates, a traveling plate with mounted rotational and translational gear trains and 2 high-torque stepper motors. The fixed position plates provide a number of functions: (1) 12 “seats” or slots (FIG. 2B) for 12 reactor vessels that allow easy removal/replacement of individual bioreactor vessels without disturbing the adjacent vessels, (2) anchors for the bioreactor vessels preventing their translation when tension is applied to the top anchor-shaft, (3) the bottom plate connected via a locking bar (FIG. 2B) and set-screw to prevent rotation of the reactor vessel as torsion is applied to the top anchor-shaft, (4) two plates to provide heat to the vessel mounts and medium as it is recirculated through the system maintaining internal vessel temperature, and (5) the top fixed plate fitted with two anti-back lash nuts against which the lead screws from the traveling plate act. The distance between plates is easily adjustable to accommodate a wide range of reactor vessel lengths.

Traveler

A traveling plate (FIG. 3A) situated above the top fixed position plate allows mounting of the two independent gear trains and provides one degree of freedom (axial) for the translation of the gear trains and the motors while constraining the other five degrees of freedom. The traveler is fitted with (1) two independent gear trains, (2) 4 self-aligning recirculating ball linear bearings for traveler translation, and (3) a motor mount plate allowing both stepper motors to engage their specific gear trains.

Drive Mechanisms

The gear trains, when driven by their specific stepper motors, independently control rotational and translational deformation. The rotational gear train includes 12×5.08 cm diameter spur gears (72 pitch) in a 1:4 ratio with the stepper motor. Each gear is aligned co-axially with the reactor vessel seated below. Each gear sits on top of a bronze thrust bearing and attaches to a 6.35 cm (2.5″) long precision ground 0.635 cm (0.250″) 316 S.S. rod. The rod extends from the gear, through the bearing in the traveler, and protrudes 1″ from the bottom surface of the traveler towards the reactor vessels. The rod is held in place via a 6.35 mm (0.250″) long collar pressed against the bottom of the thrust bearing. The remaining 1.91 cm (0.75″) of the rod is used to attach the anchor-shaft extending up from the reactor vessel via a coupling. The coupling provides an independent way of attaching/detaching the reactor vessel from the system during the course of an experiment

Translational deformation is produced via two precision 1.27 cm (0.5″) diameter 2.54 mm (0.1″) pitch stainless steel lead screws reacting against the anti-backlash nuts affixed to the top fixed-position plate. The lead screws are driven through a 1:3 reduction by a stepper motor. The stepper is capable of microstepping (51,200 microsteps/revolution). The combination of the motor's resolution and gear reduction results in sub-micron axial translation. When actuated, the entire mass of the traveler including the gear trains for both rotation and translation as well as the motors and the associated hardware (bearings, bushings, etc.) move relative to the top fixed plate and, hence, relative to the bioreactor vessels' bottom tissue anchors producing axial translation of the upper tissue anchor. This method allows for uncoupled axial and rotational displacements.

Motion Control Software

LPT Indexer motion control software, high torque stepper motors (50 lbs-in torque) with accompanying micro-step drivers (51,200 steps/rev) and power supply were purchased from Servo-Systems Co., Montville, N.J. Custom software to engage the LPT Indexer software was written in Visual Basic V. 6.0 to control the stepper motors. The software provides precise independent control over the rotational and linear movements. Rates for linear and rotational movement of the top tissue anchors range from zero to 6.5 inches/sec and 48.8 revolutions/sec. The software allows the user to input the forward and return rotational and linear rates, total excursion (rotational and linear), provides for an intermediate period of rest or static mode at the extreme point, a rest or static mode at the home point, and the number of repetitions. Several different cycles with varying strain regimes can be programmed and run for the duration of a experiment. A common loading regime for the culture of ligaments is shown in FIG. 3B.

Environmental Chamber and Gas Exchange

The system includes an environmental chamber with a cassette design in which each cassette houses recirculation tubing for two reactor vessels and two shared peristaltic pumps are responsible for fluid recirculation of the entire system. An alternative design incorporates the use of micro-pumps attached to each environmental chamber cassette providing independent control to each reactor vessel. The design also incorporates the cassettes as a continuous part or extension of the reactor vessel wall. In this case, a single cassette comprised of the reactor vessel housing the tissue as well as the recirculation loop and micro-pump may be “inserted” into the bioreactor system for independent control and ease of use.

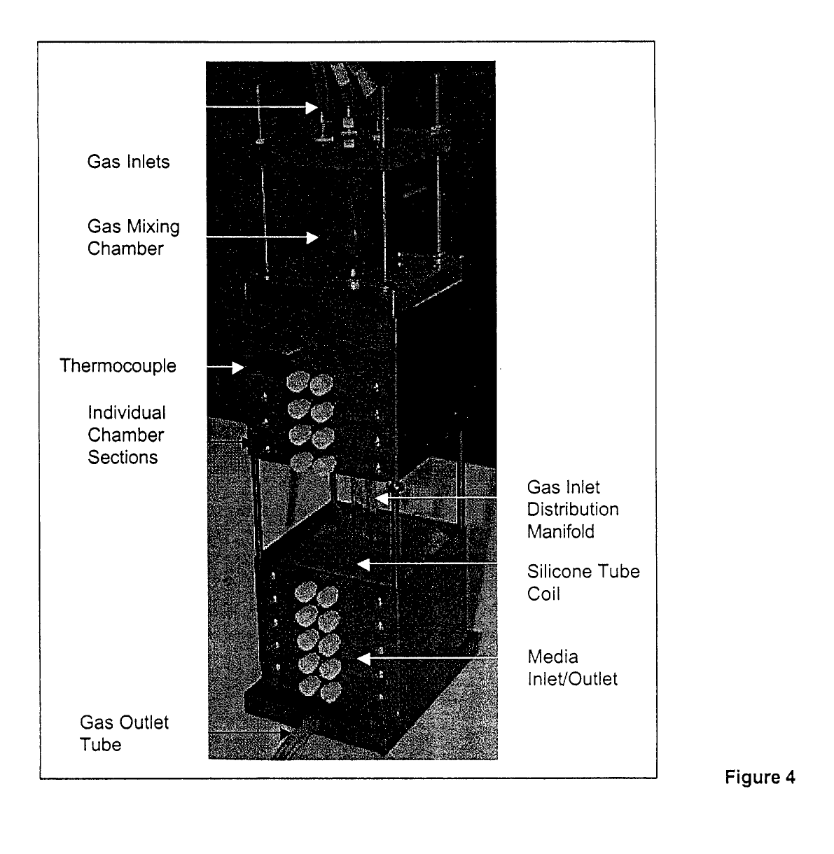

A multi-component environmental chamber (FIGS. 4A-B) was built to provide precise pH and pO₂ control to all 24 reactor vessels by silicone tubing gas exchangers (a coil of 1.83 m tubing, 1.588 mm (1/16″) I.D., 3.175 mm (⅛″) O.D., for each vessel). Environmental chamber components include (1) 12 individual chamber sections designed to house two coils per section, (2) an additional chamber to house two thermocouples to measure chamber wall and internal temperature, (3) a gas mixing plenum with 3 gas inlets, (4) a gas distribution manifold and (5) a chamber outlet port. Each chamber section provides, on opposite faces, an inlet and outlet port for recirculating vessel medium. The chamber sections were designed as independent components to provide flexibility to the system. Chamber sections are stackable and removable for quick turn-around, sterilization without entire system shutdown, and easy clean up in case of leaks. The entire chamber or individual sections, including inlet and outlet ports, can be steam sterilized. Hose dimensions and length were chosen to achieve vessel medium equilibrium with the gas environment of the environmental chamber over the range of possible medium flow rates provided by the pump system. Vessel medium is recirculated via a multi-drive microbore peristaltic pump (12 channels per pump) (Masterflex, Cole-Parmer, Vernon Hills, Ill.) through the silicone gas permeable hose at 1.7 ml/min to achieve equilibrium with chamber atmosphere; therefore, control over inlet gas flow rates can be used to adjust the dissolved gas concentrations of the reactor vessels.

Independent mass flow controllers (MKS, MA) individually control the flow rates of the three gasses into the mixing plenum. The ratiometrically correct mixture is then distributed into the chamber by the orifices of the gas inlet distribution manifold that have been sized for even distribution of the gas into the chamber taking into account the mass flow rate and the associated drag of the gas mixture. The gas permeable platinum-cured silicone hose (Cole-Parmer, Ill.) of the coils allows diffusion of the gases into the medium. Thus, pH and dissolved pO₂ are maintained at the same levels in all reactor vessels. The chamber gas outlet tube remains opened to allow gas flow through the chamber maintaining steady state. Low inlet gas flow rates were maintained such that inexpensive commercially available CO₂, O₂, and N₂ tanks will last for approximately 3 weeks.

Temperature Control System

Vessel medium temperature was maintained by heating both fixed plates of each bioreactor and the walls and inlet gas in the environmental chamber. Four CN-9000 programmable temperature control units (Omega, Stamford, Conn.) are used to maintain reactor top vessel mounts and the wall of the environmental chamber at 37° C. Four 60-watt silicone heating strips (2.54 cm×30.48 cm (1″×12″)) were used per bioreactor (2 per fixed position plate) to maintain the vessel mounts at 37° C. thus heating the medium as it enters and exits the vessel.

Biocompatible Matrix

Engineered tissues were produced by growing cells on various biocompatible matrices. Several materials including collagen type I gels and Bombyx mori silkworm silk fiber matrices were anchored, seeded with adult stem cells (hBMSCs), and grown in the system for up to 21 days. For example, acid soluble collagen type I (Sigma Type III) was neutralized, mixed with hBMSCs and gelled within the reactor vessel between anchors. Silk fiber matrices were either embedded in biocompatible epoxy adhesive (3M DP-100, McMaster-Carr, New Brunswick, N.J.) or sutured using a “whip-stitch” for attachment into the anchor-shafts. Matrices were 3 cm long between anchors. Direct clamping of the silk matrix's ends via set-screws in the anchor-shafts provided stable anchoring that can withstand >200 N of force before matrix would pull-out.

Cells

Bone marrow stromal cells, such as human bone marrow stromal cells (hBMSC), were obtained from bone marrow aspirates from human donors ≦25 years of age (Clonetics-Poietics, Walkersville, Md.). Twenty-five ml aspirates were resuspended in Dulbecco's Modified Eagle Medium (DMEM) supplemented with 10% fetal bovine serum (FBS), 0.1 mM nonessential amino acids, 100 U/ml penicillin, 100 mg/L streptomycin (P/S), and 1 ng/ml basic fibroblast growth factor (bFGF) (Life Technologies, Rockville, Md.) and plated at 8-10 μl aspirate/cm in tissue culture flasks. BMSCs were selected based on their ability to adhere to the tissue culture plastic; non-adherent hematopoietic cells were removed during medium replacement after ˜5 days in culture. Medium was changed twice per week thereafter. When primary BMSC became near confluent (12-14 days), they were detached using 0.25% trypsin/1 mM EDTA and replated at 5×10³ cells/cm². First passage (P1) hBMSCs were trypsinized and frozen in 8% DMSO/10% FBS/DMEM for future use.

Silk Matrix Cell Seeding

Frozen P1 hBMSCs were defrosted, replated at 5×10³ cells/cm² (P2), trypsinized when near confluency, and used for matrix seeding. Sterilized (ethylene oxide) silk matrices were seeded with cells in customized seeding chambers (1 ml total volume) machined in Teflon blocks to minimize cell-medium volume and increase cell-matrix contact. Seeded matrices, following a 4 hour incubation period with the cell slurry (3.3×10⁶ BMSCs/ml) were transferred into the anchor-shafts and assembled into the reactor vessel. Each vessel was loaded into the bioreactor system and infused with 30 ml of cell culture medium without bFGF.

Tissue Culture Conditions

Reactor vessels containing seeded silk matrices were loaded into a servo-hydraulic Instron 8511 (Instron Corp., Canton, Mass.), a 5 N tear load under load-control is imparted to the matrix via the top anchor-shaft, and the position held via set-screws within the top vessel mount. This sets the initial “zero” position of the scaffold before loading into the bioreactor. The reactor vessels containing the pre-stressed matrices were then loaded into the bioreactor, coupled to the rotational gear train and the mechanical regime (dynamic or static) was imparted to the developing cell-scaffold complex. Reactor vessel medium was changed batch-wise (50%/change) twice a week. pH was maintained at 7.4 and dissolved oxygen at 20% pO₂.

Bioreactor Motion Control Subsystem Characterization

As is the case in a fully utilized bioreactor, all available seats were occupied by reactor vessels during linear and angular displacement characterization. Translational hysteresis was measured using vernier calipers with resolution of 25.4 μm clamped to the bottom fixed plate of the bioreactor. Caliper jaws were situated between the anchor shafts and displacement between the top and bottom anchor shafts for vessels seated in the third and sixth positions of the bioreactor was measured for a 2 mm cyclic translation at 0.0167 Hz. A spring mounted between the traveling plate and top caliper jaw was used to ensure continuous measurement throughout the cyclic regime by keeping the caliper jaw in constant contact with the top anchor shaft. Initial displacement between shafts was set to 3 cm, the caliper zeroed and displacement at the initial home and extreme positions measured and recorded for 10 cycles. Temporal changes relative to both linear and rotational displacements were measured using a stop-watch to verify programed cyclic frequency.

Rotational hysteresis was measured on the axis of the bioreactor vessel furthest (along the gear train) from the driving axis of the stepper motor used to control rotational displacement. The bioreactor in this position suffers from the most hysteresis as hysteretical error compiles in a linear (gear train) system. The hysteresis was measured using a mirror and a coherent light source (laser). The central shaft of the reactor vessel seated in the sixth position was machined to allow placement of the reflective plane of the first surface mirror to be collinear with the axis. The laser (HeNe 3 mW) was placed such that the plane of the incident and reflected light to/from the mirror remained normal to the reflecting surface of the mirror and intersecting the axis of the bioreactor vessel. Hysteresis was measured by causing, through the motion control software and stepper motor, cyclic rotational displacement. This resulted in a linear translation of the reflected spot on a diffuse reflecting screen (e.g., white wall) approximately 3 meters from the reactor vessel and mirror; the angular measurement system resulted in 0.0268° resolution. The displacement of the reflected spot was marked relative to the initial position (zero displacement). The locations of the spot at the extreme point of rotation and following the return to the initial condition were also recorded and repeated for 9 additional cycles. In order to achieve desired resolution, available room space limited rotational displacement to 30°. Two sets of measurements (N=10 cycles per set) were performed for 20° and 30° of rotation and maximum deviations calculated.

Reactor Vessel Medium Temperature Characterization

Extensive testing was performed to ensure constant temperature about the developing tissue in the reactor vessel. Feedback temperature control of the top anchor mount and maintenance of environmental chamber wall temperature at 37° C. was used to control medium temperature about the 3 cm long silk matrix over 21 days in culture. A submersible thermocouple calibrated to 37° C. (resolution+/−0.2° C.) was used to measure temperture about the mid-section of the matrix as well as the area around where the matrix contacted the top and bottom anchor shafts. Three ports were introduced to the reactor vessel tube wall at desired locations along the length of the matrix for insertion of the thermocouple during the 21 day experiment. Measurements were taken at least twice a day and only deviations from 37° C. recorded.

pH and Dissolved pO₂ Measurements

Medium pH and dissolved O₂ were measured off-line using a blood gas analyzer (Instrumentation Laboratory 1610, Lexington, Mass.). Following 50% medium exchange, 15 ml of vessel medium extracted from the reactor vessel into a 20 ml syringe was immediately assayed for pH and pO₂.

Scanning Electron Microscopy

The matrices were harvested at timed intervals, washed with 0.2 M sodium cacodylate buffer, fixed overnight in Karnovsky fixative, dehydrated through an ethanol series and left to dry in Freon overnight. The samples were sputter-coated with Au using a Polaron SC502 Sputter Coater (Fison Instruments), and imaged at 15 keV with a JEOL JXA 840 Scanning Electron Microscope.

Characterization of Controllable Physical and Chemical Parameters

A bench-top bioreactor system (e.g., shown in FIGS. 1A&B) supported production of an engineered functional biological tissue starting from isolated cells and 3D matrices. The bioreactor system applied independent multi-dimensional complex and well-controlled mechanical strains (at <0.1 μm for translational and <0.1° for rotational strain) to the developing tissue, and precisely and accurately controlled the biochemical and fluidic environments. The bench-top system accommodates up to 24 individual reactor vessels (FIG. 2A) in two independently controlled bioreactors to permit concurrent construction of several tissues and to study a variation of strain rates and percent strain in order to identify optimal parameters for tissue development (without the need for an incubator). Enhanced flexibility was achieved through the modular bench-top design allowing concurrent but independent operation of up to 24 reactor vessels; individual reactor vessels are optionally added, replaced or withdrawn at any time during the course of an experiment without disturbing system function. Matrices were anchored into the reactor vessels to support the culture of developing tissue within a mechanically dynamic environment. Cell seeding options are improved through enhanced fluidic control (0.25-25 ml/min) utilizing perfusion through and/or sheath flow around the matrix (FIG. 1A).

Reactor vessels, via the top mount O-ring and bushing, provide a barrier to contamination while allowing the tissue cultured within the vessel to be exposed to multi-dimensional strains. Each bioreactor applies a unique programmed mechanical regime to each of its 12 housed vessels, i.e., forward and return rotational and linear rates, total is excursion (rotational and linear), intermediate static mode at the extreme point and/or the home point, the number of cycle repetitions for a specific regime, and the duration between different regimes can be controlled (FIG. 3B). The apparatus provides for an unlimited number of regimes to be programmed. The use of anti-backlash nuts and micro-step drivers combined with 1:4 (angular) and 1:3 (translation) gear reductions minimized linear system hysteresis to within the resolution of our measuring equipment (vernier calipers with resolution of 25.4 μm) and technique. Linear hysteresis over 10 cycles was measured to be less than the resolution of the caliper's at less than 25 μm or 1.25% for 2 mm of travel (FIG. 3B). Deviations between the third and sixth bioreactor seat positions from the programmed theoretical displacements were not discernable as well. Rotational hysteresis was measured to be less than 0.2° (0.36%) for a angular displacement of 30° (FIG. 3B) and was less than 0.1° for a 20° displacement over 10 cycles. It is reasonable to expect when extrapolating to 90° of rotation, hysteresis of substantially less than 1% of total displacement will result. The accuracy of the programmed cyclic frequency derived from the motion control software was verified to within +/−0.5 sec or the resolution of the stop-watch and timer.

A total of 48 reactor vessels containing cell culture medium and silk matrices were run for up to 21 days under 90° rotational and 2 mm translational deformations at 0.0167 Hz. Of the 48 vessels, only one became contaminated during culture due to a leak on the outlet port. Feedback temperature control of the top anchor mount to 37° C. and the maintenance of wall temperature at 37° C. in the environmental chamber resulted in constant temperature within the resolution of the device (+/−0.2° C.) about the 3 cm long silk matrix housed in the reactor vessel. A submerged wire thermocouple place in the middle and at the two ends of the matrix anchored in the reactor vessel demonstrated that constant temperature was maintained at 37+/−0.2° C. (mean+/−standard deviation) about the silk matrix over the 21 days of operation.

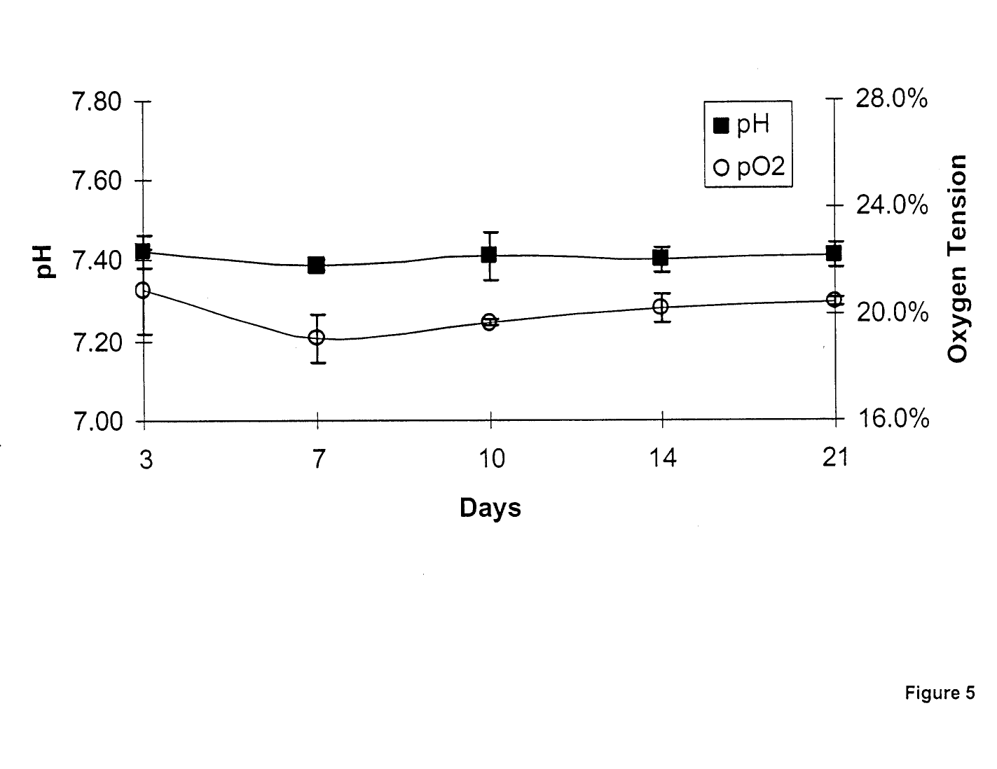

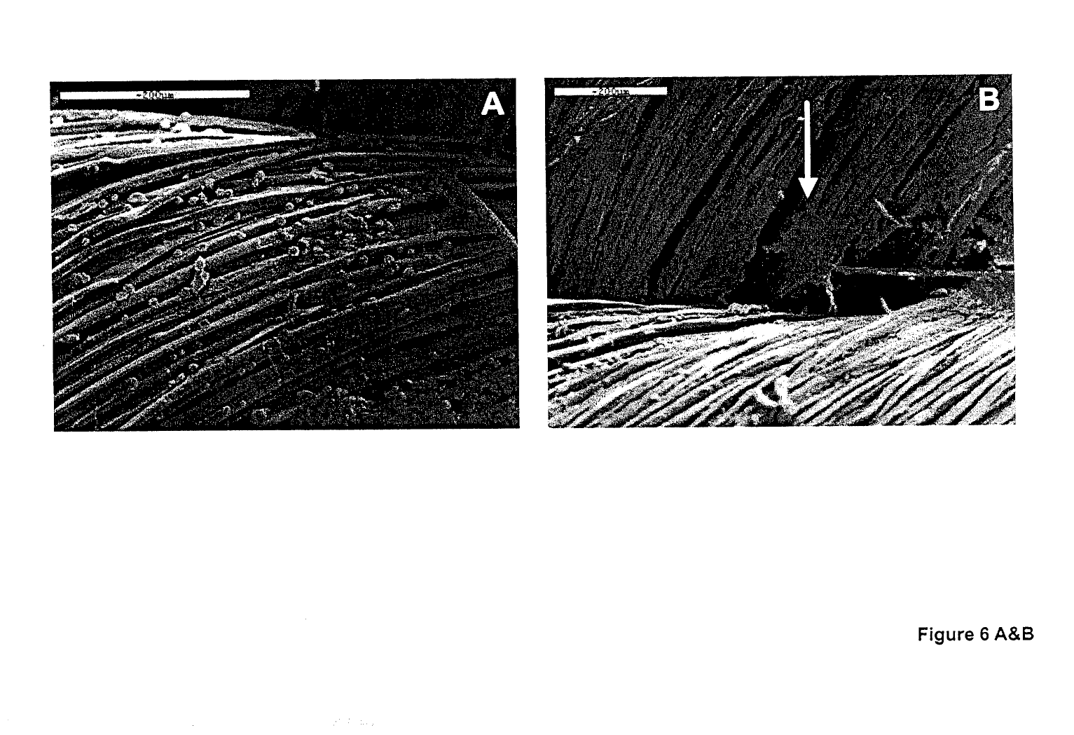

The system functioned by recirculating (via the peristaltic pumps) cell culture medium through a closed loop consisting of the reactor vessel, C-Flex, and silicone tubing from the reactor vessel through the tubing and environmental chamber and back into the reactor vessel. Fine adjustment and control of medium pH and pO₂ was achieved by controlling the gas flow rates into the environmental chamber (FIG. 4); the environmental chamber provided control over dissolved oxygen tension (between 0%-95%±1%) by achieving equilibrium with the recirculating medium. Twenty-four reactor vessels containing silk matrices seeded with hBMSCs maintained a constant 7.4. pH and 20% pO2 over 21 days in culture (FIG. 5). The bioreactor system supported hBMSCs spreading and growth on silk fiber matrices as demonstrated by SEM at a flow rate of 2.5 ml/min, pH of 7.4+/−0.02 and pO₂ of 20+/−0.5% (FIGS. 6A-B).

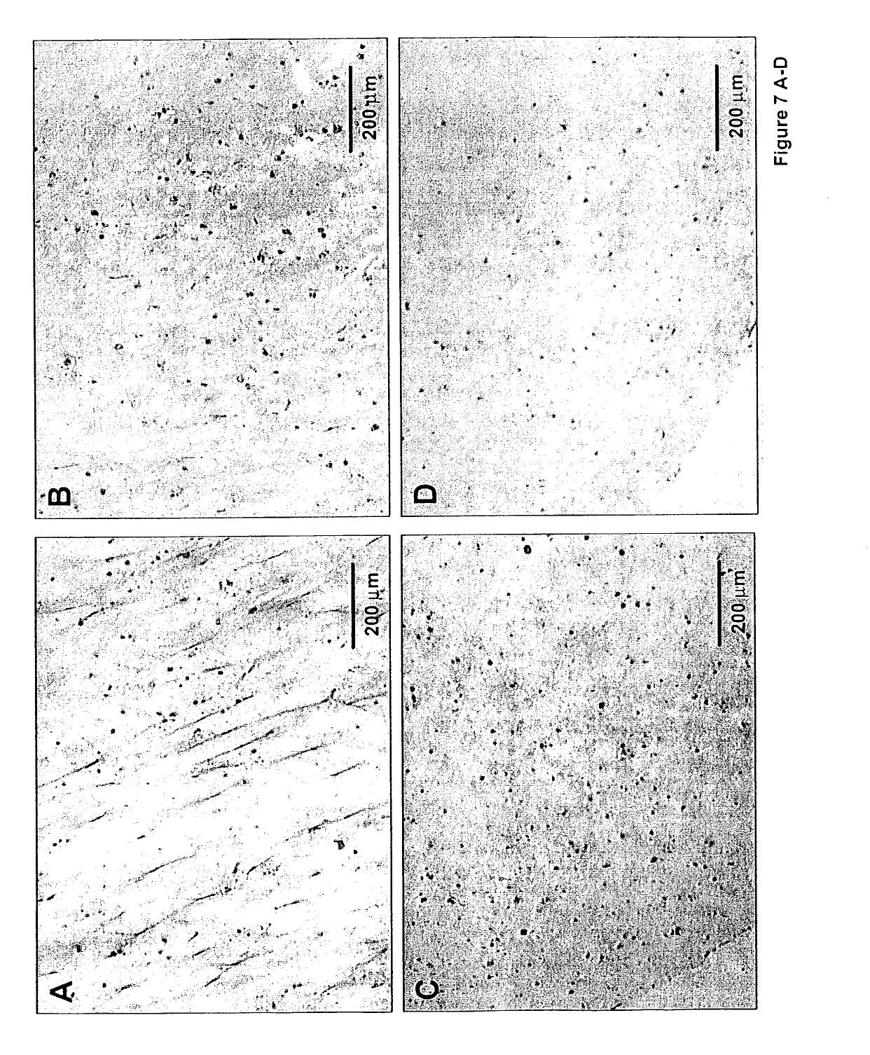

Mechanical stimulation (cyclic rotation (90°) and translation (2 mm) at 0.0167 Hz without rest at home or extreme positions) applied up to 21 days via the bioreactor induced the elongation of bone marrow stromal cells seeded within a collagen type 13-dimensional gel and increased cross-sectional cell density ˜2-fold as compared to statically grown tissue (FIGS. 7A-D). Directed multi-dimensional strains that mimic the physiological environment of the ACL specifically direct the differentiation of BMSCs towards the ligament lineage.

The relevance of applying both translational and rotational strains to developing ACL tissue is seen in the structure-function relationship of the ligament. The ACL has a unique helical fiber organization and structure to perform its stabilizing functions. The mode of attachment to bone and the need for the knee joint to rotate ˜140° (extension/flexion) results in a 90° twist of the ACL major fiber bundles developing a helical organization. In full extension, individual fibers of the ACL are attached anterior-posterior and posterior-anterior from the tibia to the femur in the sagittal plane of the knee. This helical geometry allows the individual ACL fiber bundles, during knee joint flexing, to develop a flexion axis about which each individual fiber bundle or fascicle twists thereby remaining isometric in length. Fiber bundle isometry allows the ACL to equally distribute load to all fiber bundles, maximizing its strength. It is this unique structure-function relationship that allows the ACL to sustain high loading through all degrees of knee joint extension and flexion. A tissue engineered prothesis exposed to physiologically relevant translational and rotational strains will develop a structure suitable for function following implantation in vivo. Without exposure to physiologically relevant rotational strains (e.g., only translation), an in vitro ligament engineering would fail to support the development of a helical structure capable of effectively distributing load throughout the tissue placing the prosthesis at a higher risk for rupture. While rotational strain alone acts to translate individual fibers organized in a helical geometry, translational strains are needed to control fiber pitch angle and mimic anterior draw loads typically stabilized by the ACL. Thus, the combination of both translational and rotational strains is needed for ACL tissue engineering.

The bench-top bioreactor system supported cell and tissue growth on the silk matrices while providing a multitude of biochemical and mechanical parameters for manipulation. Enhanced mechanical, biochemical and fluidic control systems of the bioreactor provide for accurate and precise control of the environment for developing tissue. The bench-top design increases system flexibility as well as the total number of reactor vessels that can be cultured in parallel. Optimal biochemical and mechanical parameters (e.g., oxygen tension, strain, strain rate, medium flow rate, type of flow) to induce ligament-specific differentiation of hBMSCs seeded on silk matrices are currently being identified. The bioreactor system optionally includes a force monitoring system and enhanced fluid handling capabilities (i.e., for medium exchange or the programmed introduction of relevant biochemical factors).

Advantages

The bioreactor system described herein offers numerous advantages over existing devices or systems. For example, the system is bench-top, i.e., it does not require housing within a tissue culture incubator and computer controlled with fluidic, temperature, gas and multidimensional strain control, as well as direct monitoring of forces on the tissue. Thus, the adjustments in environmental conditions are iterative, e.g., strain is applied and force on the tissue monitored to allow precise control of environmental factors to which the developing tissue is exposed.

Unlike earlier designs, the improved bioreactor maintains constant hydrostatic pressure and exerts strain on a tissue without a change in volume of the media with the reactor vessel. For example, the bioreactor system is differentiated from and provides additional features compared to the one described in U.S. Pat. No. 6,287,340. The improved reactor vessel design contains options for fluid/medium recirculation, allowing for the maintenance of constant pH throughout the reactor vessel. In some earlier systems, batch diffusion was carried out through a vent in the top of the reactor system. The improved system provides recirculation options of either perfusion flow through the tissue and/or sheath flow about the outside of the tissue (e.g., around the outer walls of the tissue), while still being able to mechanically strain the tissue specimen.

The improved bioreactor includes a means for direct coupling of tissue (cell-seeded matrix) to the reactor device and a means for exerting stretching and twisting forces upon the developing tissue. The improved design of the reactor vessel can encompass an o-ring seal, but also allows for a novel bellow design that diminishes friction between the moving anchor shaft and the reactor vessel housing (or top mount). The bellow design allows for tension and compression force/load monitoring down to 0.1 Newtons of force. The improved design of the reactor vessel and anchor-shaft allows direct measurement of the force a tissue exerts by placing a tension-compression load cell between the reactor vessel and its seat.

The improved bioreactor system incorporates a bench top design utilizing an environmental chamber to control for CO₂ and O₂ dissolved gas levels. In contrast to earlier simple reservoir medium systems, the improved system includes feedback/control features for accurate and precise control over pH and O₂. The system permits variation of one or more (e.g., either N₂, O₂, CO₂) gases independently to achieve a customized experimental system. Earlier systems could only function by being housed in an incubator where there exists no means of controlling O₂ gas concentration in the medium. Furthermore, the presence of an environmental chamber and the ability to recirculate medium eliminates concentration gradients in the reactor vessel and about the cultured tissue.

The improved bioreactor system also permits accurate force monitoring of the tissue. Unlike earlier systems, e.g., U.S. Pat. No. 6,121,042, the improved bioreactor system described herein does not include a magnet or subject a growing bioengineered tissue to a magnetic field (which may adversely affect growth and differentiation of cells on a matrix). Earlier systems employed a magnet or a bellows (attached to a substrate or as part of the reactor vessel) to apply force. Drawbacks of such systems include aberrant cell growth and fluctuations in fluid pressure and volume (respectively), which may also adversely affect cell growth. The improved system maintains constant pressure and constant volume while strain is being applied to the tissue. An o-ring/piston mechanism of some earlier systems creates friction, thereby reducing the accuracy by which force can be measured. The improved reactor vessel design permits both the (1) the direct coupling of an anchor shaft to the tissue specimen under culture and (2) the direct measurement of force at the tissue without requiring backing out force due to friction associated with an o-ring or the need to over come pressure differentials associated with or stretching a bellows in which the tissue specimen is housed. No prior systems accomplish both aspects in a single design.

The improved bioreactor system, through the use of the motion control plate and two independently controlled motors incorporating both linear and thrust bearing designs, allows accurate and precise control for both translational and rotational strains, either independently or in combination. Unlike earlier systems, the improved device includes both a means by which both tension and torsional forces may be applied to the tissue and a means for measuring directly the resultant force of the tissue.

The improved system overcomes many of the drawbacks of earlier systems. For example, a magnetic actuator in earlier systems preclude direct attachment between tissue and actuator and thus, precludes direct force monitoring. Systems that employ a piston permit direct coupling to tissue, but actuation creates pressure differentials as well as an inability to directly measure the force at the tissue due to the o-ring sealing mechanism. Some earlier systems include a bellow, but the tissue was housed within the bellows. With such bellows systems, translation (stretching/compressing) created a pressure differential leading to additional forces to which the tissue is exposed and aberrant cell growth. Systems with flexible membrane chamber require changing hydrostatic pressure to strain the tissue and cannot therefore be decoupled. Variations in fluid volume and hydrostatic pressure are undesirable, leading to improper growth and differentiation of cells and development of a functional tissue.

The improved system has the added advantages of accomplishing the combination of (1) the independent, but concurrent control of both translational and rotational strains, (2) direct attachment to the tissue specimen to ensure the strains programs are the strains that the tissue is forces to undergo, (3) the ability to monitor force (either applied to or resulting from the tissue), and (4) control for fluid flow both perfused through the center of the tissue along the longitudinal axis or about the tissue body, without creating pressure differentials in the system, which may induce unwanted or uncontrolled environmental signals to the culturing tissue.

Other embodiments are within the scope and spirit of the invention. For example, referring to FIG. 1A, numerous mechanisms and configurations are used to translate and rotate the shaft relative to the reactor vessel. For example, a motor can be mounted to a fixed plate and to the shaft, or to a fixed wall and the reactor vessel, or to the vessel and the shaft (and possibly to a housing wall).

What is claimed is:

1. A bioreactor system for producing bioengineered tissue using a tissue matrix, the system comprising:
a reactor vessel configured to define an interior vessel volume to contain the tissue matrix and a tissue culture for providing nutrients to the tissue matrix, the vessel having a first portion configured to couple to the tissue matrix at a location opposite a hole defined by a second portion of the vessel, the first portion providing a first vessel passageway in fluid communication with the interior volume of the vessel and a first vessel port;
a shaft configured to move within the hole defined by the second portion of the reactor vessel and to couple to the tissue matrix, the shaft providing a shaft passageway disposed to be in fluid communication with a shaft port and with an interior volume of the tissue matrix when the matrix is coupled to the shaft; and
a pump coupled in series between the shaft port and the first vessel port and configured to produce a flow of the culture through the first portion of the vessel, the shaft, and the interior volume of the tissue matrix.

2. The system of claim 1, wherein said tissue is a ligament or tendon.

3. The system of claim 1, wherein said matrix is tubular.

4. The system of claim 1, wherein the vessel is configured such that the interior vessel volume has a substantially constant volume.

5. The system of claim 4, wherein the vessel has a substantially rigid exterior.

6. The system of claim 1, wherein the first portion of the vessel provides at least one second passageway in fluid communication with the interior vessel volume, the second portion of the vessel provides at least one third passageway in fluid communication with the interior vessel volume, and wherein the pump is coupled to the vessel to be in fluid communication with the at least one second passageway and the at least on third passageway to produce a flow of the culture in the interior vessel volume exterior to the tissue matrix.

7. The system of claim 1, further comprising a tension load monitor including first and second couplings, the first coupling being coupled to the reactor vessel in a fixed relationship to the location of the first portion of the reactor vessel that is configured to couple to the tissue matrix.

8. The system of claim 1, further comprising a culture chamber coupled in series with the first vessel port, the shaft port, and the pump, and configured to produce the culture such that the culture has a desired ratio of N₂, O₂, and CO₂ in the chamber.

9. The system of claim 1, wherein the shaft is coupled to the reactor vessel such that the shaft can slide within the hole in the second portion of the vessel and rotate with respect to the vessel substantially free of frictional forces between the shaft and the vessel while inhibiting exogenous items from entering the interior vessel volume.

10. The system of claim 1, further comprising a bellows coupled to the shaft and to the reactor vessel.

11. The system of claim 1, further comprising moving means for at least one of translating and rotating the shaft relative to the reactor vessel.

12. The system of claim 11, wherein the moving means is configured to translate and rotate the shaft relative to the reactor vessel concurrently.

13. A bioreactor system for producing bioengineered tissue using a tissue matrix, the system comprising:
a reactor vessel configured to define an interior vessel volume to contain the tissue matrix and a tissue culture for providing nutrients to the tissue matrix, the vessel having a first portion configured to couple to the tissue matrix at a location opposite a hole defined by a second portion of the vessel;
a shaft configured to move within the hole defined by the second portion of the reactor vessel and to couple to the tissue matrix; and
a tension load monitor including first and second couplings, the first coupling being coupled to the reactor vessel in a fixed relationship to the location of the first portion of the reactor vessel that is configured to couple to the tissue matrix.

14. The system of claim 13, wherein said tissue is a ligament or tendon.

15. The system of claim 13, wherein said matrix is tubular.

16. The system of claim 13, wherein the shaft is coupled to the reactor vessel such that the shaft can slide within the hole in the second portion of the vessel and rotate with respect to the vessel substantially free of frictional forces between the shaft and the vessel while inhibiting exogenous items from entering the interior vessel volume.

17. The system of claim 16, further comprising a bellows coupled to the shaft and to the reactor vessel.

18. The system of claim 13, further comprising moving means for at least one of translating and rotating the shaft relative to the reactor vessel.

19. The system of claim 18, wherein the moving means is configured to translate and rotate the shaft relative to the reactor vessel concurrently.

20. The system of claim 13, further comprising a housing, wherein the second coupling of the load monitor is coupled to the housing.

21. A bioreactor system for producing bioengineered tissue using a tissue matrix, the system comprising:
a reactor vessel having a substantially rigid exterior and configured to define a substantially constant-volume interior volume to contain the tissue matrix and a tissue culture for providing nutrients to the tissue matrix, the vessel having a first portion configured to couple to a first end of the tissue matrix at a location opposite a hole defined by a second portion of the reactor, the first and second portions providing at least one first and second reactor passageway, respectively, in fluid communication with the interior volume of the vessel, the at least one first reactor passageway being in fluid communication with at least one reactor intake port and the at least one second reactor passageway being in fluid communication with at least one outlet port;
a shaft configured to slide within the hole defined by the second portion of the reactor vessel and to couple to a second end of the tissue matrix, the shaft providing a shaft passageway disposed to be in fluid communication with a shaft intake port, and in fluid communication with an interior volume of the tissue matrix when the matrix is coupled to the shaft;
a load monitor coupled to the reactor vessel in a fixed relationship to the first portion of the reactor vessel;
a culture chamber having an input coupled to the at least one outlet port and configured to produce the culture having a desired ratio of N₂, O₂, and CO₂ in the chamber, the chamber being further coupled to the at least one reactor intake port and the shaft intake port; and
a pump coupled between the at least one outlet port and the at least one reactor intake port and the shaft intake port and configured to produce a flow of the culture from the chamber, through the shaft and the second portion of the reactor vessel, into the interior volume of the vessel and the interior volume of the tissue matrix, through the first portion of the reactor vessel, and back to the chamber;
wherein the shaft is coupled to the reactor vessel such that the shaft can slide within the hole in the second portion of the vessel and rotate with respect to the vessel substantially free of frictional forces between the shaft and the vessel while inhibiting exogenous items from entering the interior volume of the reactor vessel.
